(19) United States
(12) Patent Application Publication
(10) Pub. No.: US 2006/0058771 A1
(43) Pub. Date: Mar. 16, 2006

(54) Absorbent article

(76) Inventor: Carol Erdman, West Chester, PA (US)

Correspondence Address: Mark S. Leonardo, Esq.;Brown Rudnick Berlack Israels LLP, One Financial Center, Boston, MA, 02111, US

(21) Appl. No.: 10/941,375

(22) Filed: Sep. 15, 2004

(51) Int. Cl. A61F 13/15

(52) U.S. Cl. 604/390; 604/389

(57) ABSTRACT

The present disclosure provides for a fastener for use with an absorbent article having a first end and a second end and a disengagement portion disposed intermediate the first and second ends. Actuation of the disengagement portion in the direction of the first end disengages the fastener from the absorbent article. In contrast, actuation of the disengagement portion in the direction of the second end does not disengage the fastener from the absorbent article. The disengagement portion is attached generally to a mid-portion of the fastener and may include a finger lift portion.

### BACKGROUND

1. Technical Field

The present disclosure generally relates to disposable absorbent articles, and more particularly, to disposable absorbent articles with improved fasteners.

2. Background of the Related Art

Absorbent articles such as, for example, disposable diapers, adult incontinent pads, sanitary napkins, pantiliners, incontinent garments, etc. are generally worn, in cooperation with garments and disposed against a body surface by infants or adult incontinent individuals. The absorbent article is employed to collect and absorb body fluid discharge, such as, for example, blood, menses, urine, aqueous body fluids, mucus and cellular debris. For example, the absorbent article may be disposed between the legs of an individual adjacent a crotch area. The absorbent article is positioned with a garment and drawn into engagement with a body surface of the crotch area to collect fluid discharge.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as wood pulp, for acquiring and storing fluid discharge.

In addition, fastening of the absorbent articles with an individual requires the use of fasteners or ear members and closure tabs that extend laterally from the body of the absorbent article. In addition, the closure tabs typically include mechanical closure material, for example, hook and/or loop material, adhesive tape or the like. For example, in typical diaper-type garments, the garment is affixed to a wearer by attaching one or more of the closure tabs that extend across the wearer's hips to hold the back and front halves of the garment to one another.

Some of these absorbent articles, however, suffer from performance drawbacks including that the article can be easily unfastened by a wearer, fore example, a child. Generally, children between the age of about 18 months to about 36 months begin to show a curiosity with their diaper that eventually leads to their playing with the conventional fasteners. In this age range, children have the dexterity and strength to unfasten the fastener and thus remove the diaper at an inopportune time.

One typical absorbent article 104 is shown in FIGS. 1A-1C, and includes a fastener 100 having a lift region 102 disposed at a far end thereof. There is concern that it is easy for a child to pull the lifting region 102 in a direction B and thus unfasten the fastener 100.

It would therefore be desirable to overcome the disadvantages and drawbacks of the prior art by providing an absorbent article including unique fasteners that employ a method of detachment that is less likely of being inadvertently removed by a child wearer.

### SUMMARY

Accordingly, an absorbent article is disclosed that includes an absorbent article with a child resistant closure system. Objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure consisting of its constituent parts, constructions, arrangements, combinations, steps and improvements herein shown and described.

In one particular embodiment, the absorbent article, which may be a diaper, includes a backsheet, a topsheet, and an absorbent core disposed therebetween. The diaper extends from a first end to a second end along a longitudinal centerline and includes a fastener attached to the first end and being engageable with the second end for disposing the absorbent article about a subject wearer.

The present disclosure provides for a fastener for use with an absorbent article having a first end and a second end and a disengagement portion disposed intermediate the first and second ends. Actuation of the disengagement portion in the direction of the first end disengages the fastener from the absorbent article. In contrast, actuation of the disengagement portion in the direction of the second end does not disengage or greatly hampers the detachment of the fastener from the absorbent article. The disengagement portion is attached generally to a mid-portion of the fastener and may include a finger lift portion.

The fastener may include a substrate having a first side and a second side wherein the disengagement portion protrudes from the first side. The width of disengagement portion may be substantially equal to that of the width of the fastener and may be formed from a folded portion of the fastener or separate finger lift portion that is attached to the fastener.

The present disclosure provides for a diaper including a backsheet, a topsheet, and an absorbent core disposed therebetween. The diaper extends from a front end to a back end and includes one or more fasteners attached to the back end engageable with the front end for disposing the diaper about a subject.

The fastener includes at its mid-length a finger lift for disengaging the fastener from the front end when the finger lift is actuated in an inward direction. Actuation of the finger lift may include lifting or pulling the finger lift in an inward direction for disengaging the fastener from the front end of the diaper. Actuation of the finger lift in an outward direction does not disengage or greatly hampers the detachment of the fastener from the front end of the diaper.

The fastener may include one or more substrates having a first side and a second side wherein the finger lift is generally attached to the first side of the one or more substrates. The finger lift may also be formed by a folded portion of the fastener and include an edge portion that has a non-straight or wavy configuration.

The present disclosure also provides for a diaper including a backsheet, a topsheet and an absorbent core disposed therebetween. The diaper extends from a front end to a back end and includes a fastener distally attached to the back end and being engageable with the front end for disposing the diaper about a subject.

The fastener includes a first substrate having a proximal end attached to the back end of the diaper and a second substrate attached to the first substrate. The first or second substrates may also include a distal portion for engaging the front end of the diaper and a centrally disposed portion forming a means for disengagement of the fastener from the front end of the diaper. The distal portion may also include hook material and a distal edge that extends beyond the hook material and is co-extensive with the hook material. An edge portion of the means of disengagement may include a non-straight or wavy configuration.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1A is a partial plan view of a fastener for use with an absorbent article according to the prior art;

FIG. 1B is a partial side view of the fastener of FIG. 1A;

FIG. 1C is a perspective view of a fastener and absorbent article according to the prior art;

FIG. 2 is a plan view of an absorbent article according to the present disclosure;

FIG. 3 is a partial side view of the absorbent article according to FIG. 2;

FIG. 4 is a partial side view of a fastener for use with an absorbent article according to the present disclosure;

FIG. 5 is a partial plan view of a fastener for use with an absorbent article according to the present disclosure;

FIG. 6 is a partial bottom view of the fastener of FIG. 5;

FIG. 7 is a partial side view of a fastener for use with an absorbent article according to the present disclosure;

FIG. 8 is a partial side view of a fastener for use with an absorbent article according to the present disclosure; and

FIG. 9 is a perspective view of an absorbent article according to the present disclosure.

### DETAILED DESCRIPTION OF EXEMPLARY EMBODIMENTS

The exemplary embodiments of the absorbent article and methods of use disclosed are discussed in terms of fluid absorbent articles, and more particularly, in terms of an absorbent article including fasteners that cooperate to improve attachment and fit. The presently disclosed absorbent article is contemplated to be employed with, for example, disposable diapers, training pants, adult incontinent products and other incontinent garments.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. As used herein, the term “absorbent article,” “absorbent garment” or “garment” refers to garments that absorb and contain exudates, and more specifically, refers to garments that are placed against or in proximity to the body of the wearer to absorb and contain the various exudates discharged from the body.

A non-exhaustive list of examples of absorbent garments includes diapers, diaper covers, disposable diapers, training pants, feminine hygiene products and adult incontinence products. The term garment includes all variations of absorbent garments, including disposable absorbent garments that are intended to be discarded or partially discarded after a single use and unitary disposable absorbent garments that have essentially a single structure. As used herein, the term “diaper” refers to an absorbent garment generally worn by children and incontinent persons about the lower torso.

The claims are intended to cover all of the foregoing classes of absorbent garments, without limitation, whether disposable, unitary or otherwise. These classifications are used interchangeably throughout the specification, but are not intended to limit the claimed invention. The invention will be understood to encompass, without limitation, all classes of absorbent garments, including those described above.

Absorbent garments and diapers may have a number of different constructions. In each of these constructions it is generally the case that an absorbent core is disposed between a liquid pervious, body-facing topsheet and a liquid impervious, exterior facing backsheet. In some cases, one or both of the topsheet and backsheet may be shaped to form a pant-like garment. In other cases, the topsheet, backsheet and absorbent core may be formed as a discrete assembly that is placed on a main chassis layer and the chassis layer is shaped to form a pant-like garment. The garment may be provided to the consumer in the fully assembled pant-like shape or may be partially pant-like and require the consumer to take the final steps necessary to form the final pant-like shape, such as by fastening one or more fasteners or fastener tabs.

In the case of some diapers and most adult incontinent products, the garment often is provided fully formed with factory-made side seams and the garment is donned by pulling it up the wearer's legs. In the case of most diapers, wherein, for example, a child lies on his or her back, a caregiver usually places the diaper between the child's legs, pulls the front end of the diaper up between the legs and then attaches one or more closure tabs to the front waist region of the diaper, thereby forming a pant-like structure. For clarity, the present invention is described herein only with reference to a diaper-type garment in which the topsheet, backsheet and absorbent core are assembled into a structure that forms a pant-like garment when secured on a wearer using fastening devices, although the invention may be used with any other type of absorbent garment that may benefit from the use or addition of fastener tabs.

The following discussion includes a description of the absorbent article in accordance with the present disclosure. Reference will now be made in detail to the exemplary embodiments of the disclosure, which are illustrated in the accompanying Figures.

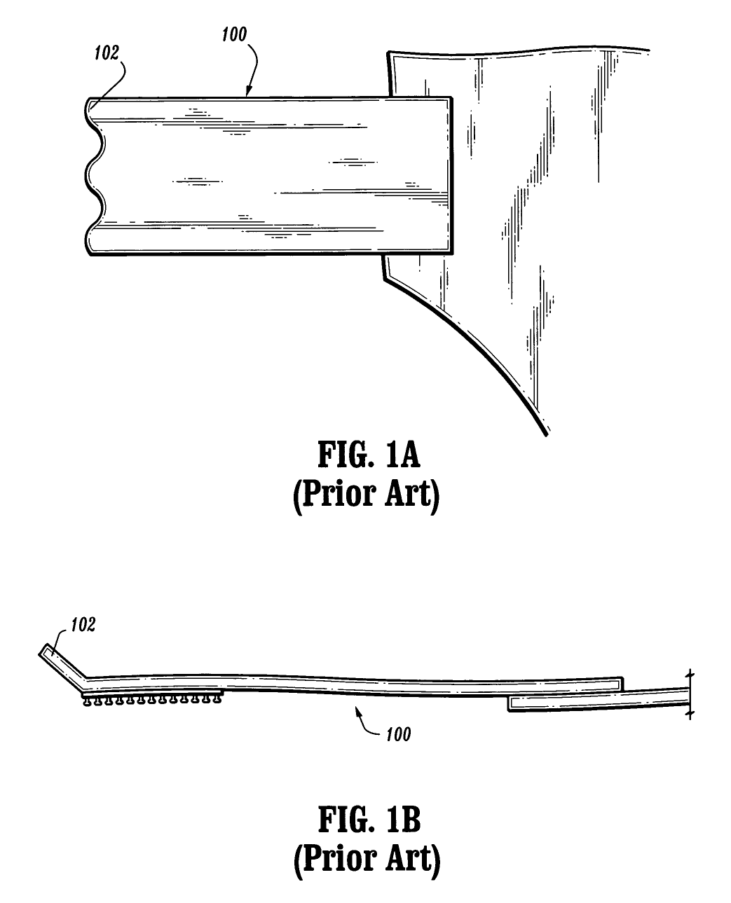

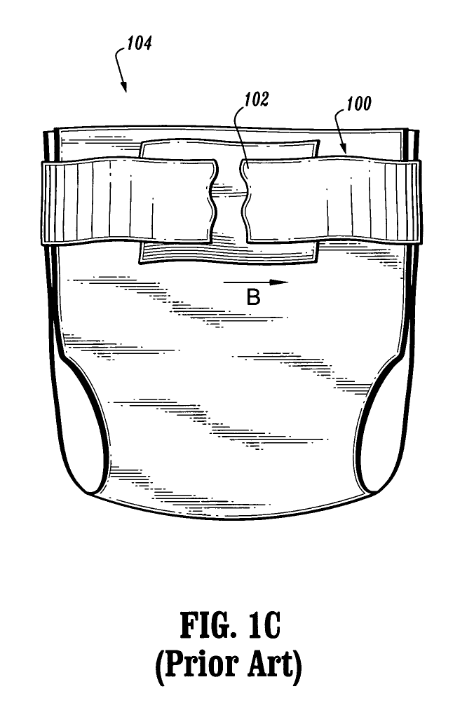

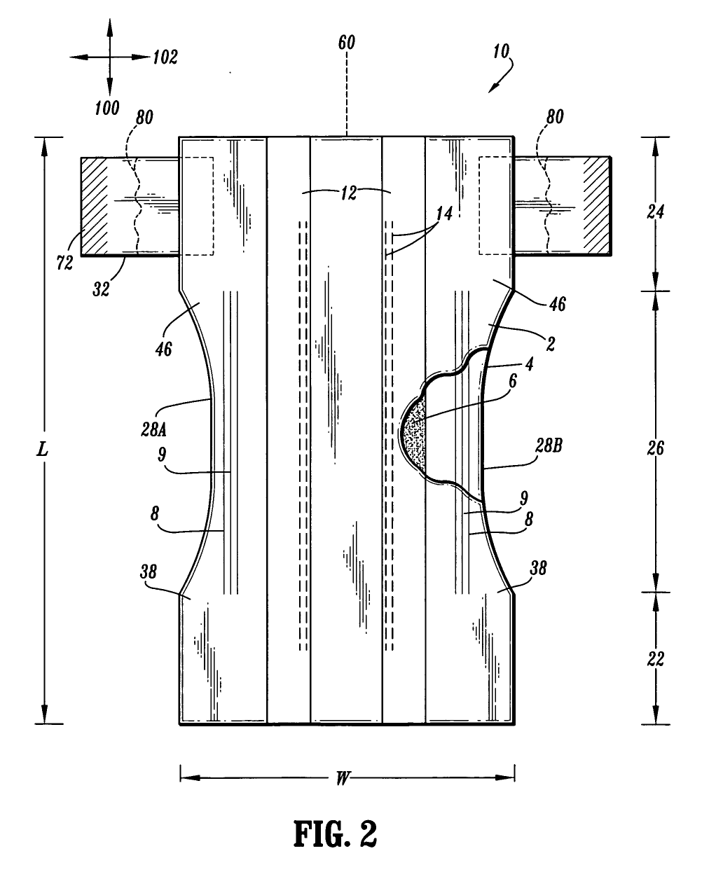

Turning now to the Figures, wherein like components are designated by like reference numerals throughout the several views. Referring to FIGS. 2-9, there is illustrated an absorbent article, constructed in accordance with the principles of the present disclosure, such as, for example diaper 10. An improved diaper 10 is provided, as compared with an example of the prior art shown in FIGS. 1A-1C. In FIG. 2, diaper 10 is shown in a relaxed condition with the effects of the elastics 8, 14 removed for purposes of clarity in the description. The diaper 10 chassis generally has an hourglass shape. The chassis generally can be defined in terms of a front waist region 22, a back waist region 24 and a crotch region 26. Those skilled in the art will recognize that “front” and “back” are relative terms, and these regions may be transposed without departing from the scope of the present invention. Alternatively, the diaper chassis can be configured in a generally rectangular shape or in a “T” shape. The diaper preferably comprises a topsheet 2, a backsheet 4, which may be either a different size than the topsheet 2 or may be substantially coterminous with the topsheet 2 and an absorbent core 6 disposed between at least a portion of the topsheet 2 and backsheet 4. Throughout this description, the terms “topsheet” and “backsheet” denote the relationship of these materials or layers with respect to the absorbent core 6. It is understood that additional layers may be present between or beyond the absorbent core 6 and the topsheet 2 and backsheet 4 and that additional layers and other materials may be present on the side opposite the absorbent core 6 from either the topsheet 2 or the backsheet 4. A pair of leg openings 28a, 28b extend along at least a portion of the crotch region 26 and one or more pairs of leg elastics 8 (three pairs are shown in FIG. 2) may be disposed to extend adjacent to leg openings 28a, 28b. Of course, in other embodiments, the number of leg elastics 8 may be increased, decreased or omitted altogether.

The diaper 10 generally has a longitudinal direction 100 that extends generally parallel to the front-to-back axis of a wearer, and a lateral direction 102 that extends generally parallel to the side-to-side axis of a wearer. The diaper 10 generally is symmetrical about a longitudinal centerline 60, but also may have asymmetrical components or shapes. The terms “inward” or “proximal,” and “outward” or “distal,” as used herein, refer to positions generally along the lateral direction 102, with “inward” locations being located closer to the longitudinal centerline 60 than “outward” locations.

The diaper may further include a waste containment system in the form of waste containment flaps 12 (also known as inner leg gathers (“ILG's”), unitary leg gathers or standing leg gathers) and one or more pairs of elastics 14. Waste containment flaps 12 preferably extend from the front waist region 22 to the back waist region 24 along opposite sides of the longitudinal center line 60 of the diaper 10 or alternatively only along a portion thereof. The front waist region 22 and rear waist region 24 preferably include side panels or ear portions 38, 46, extending outward from the leg openings 28a, 28b to provide the garment 10 with an hourglass shape.

A variety of backsheet and topsheet constructions and materials are available and known in the art and the invention is not intended to be limited to any specific materials or constructions of these components. The backsheet 4 may be made from any suitable pliable liquid-impervious material known in the art. Typical backsheet materials include films of polyethylene, polypropylene, polyester, nylon and polyvinyl chloride and blends of these materials. For example, the backsheet can be comprised of a pigmented polyethylene film having a thickness in the range of 0.02-0.04 mm. The moisture-pervious topsheet 2 can be made of any suitable relatively liquid-pervious material known in the art that permits passage of liquid therethrough. Non-woven topsheet materials are exemplary because such materials readily allow the passage of liquids to the underlying absorbent core 6. Examples of suitable topsheet materials include non-woven spunbond or carded webs of polypropylene, polyethylene, nylon, polyester and blends of these materials.

The backsheet 4 and the topsheet 2 preferably are “associated” with one another. The term “associated” encompasses configurations whereby the topsheet 2 is directly joined to the backsheet 4 by affixing the topsheet 2 directly to the backsheet 4, and configurations whereby the topsheet 2 is indirectly joined to the backsheet 4 by affixing the topsheet 2 to intermediate members which in turn are affixed to the backsheet 4. While the backsheet 4 and topsheet 2 in the present disclosure have substantially the same dimensions, they may also have different dimensions.

In addition, the backsheet 4 may be covered with a fibrous, non-woven fabric layer such as is disclosed, for example, in U.S. Pat. No. 4,646,362, which is incorporated herein by reference in its entirety and in a manner consistent with the present invention. Materials for such a fibrous outer liner include a spun-bonded non-woven web of synthetic fibers such as polypropylene, polyethylene or polyester fibers; a non-woven web of cellulosic fibers, textile fibers such as rayon fibers, cotton and the like, or a blend of cellulosic and textile fibers; a spun-bonded non-woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers.

The backsheet 4 may comprise multiple panels, such as, for example, three panels wherein a central poly backsheet panel is positioned adjacent the absorbent core while distal non-woven breathable side backsheet panels are attached to the side edges of the central poly backsheet panel. The backsheet may also be formed from microporous poly coverstock for added breathability. In other embodiments, the backsheet may be a laminate of several sheets. The backsheet may further be treated to render it hydrophilic or hydrophobic, and may have one or more visual indicators associated with it, such as labels indicating the front or back of the diaper or other characters or colorations. The present invention is not limited to any particular backsheet 4 material or construction.

The topsheet 2 may be formed from one or more panels of material and may comprise a laminated sheet construction. In the embodiment of FIG. 2, the topsheet comprises one panel across the entire width of the diaper and is formed from a liquid-pervious material that is either hydrophobic or hydrophilic. Topsheet 2 may be made from any number of materials, including synthetic fibers (e.g., polypropylene or polyester fibers), natural fibers (e.g., wood or cellulose), apertured plastic films, reticulated foams and porous foams to name a few. One preferred material for topsheet 2 is a cover stock of single ply non-woven material which may be made of carded fibers, either adhesively or thermally bonded, perforated plastic film, spunbonded fibers, or water entangled fibers, which generally weigh, for example, from 0.3-0.7 oz./yd² and have appropriate and effective machine direction and cross-machine direction strength suitable for use as a baby diaper cover stock material, as are known in the art. Topsheet 2 preferably extends from substantially the front waist region 22 to the back waist region 24 or a portion thereof, and includes a surfactant (at least along the core area) to aid in the passage of exudates to the core 6.

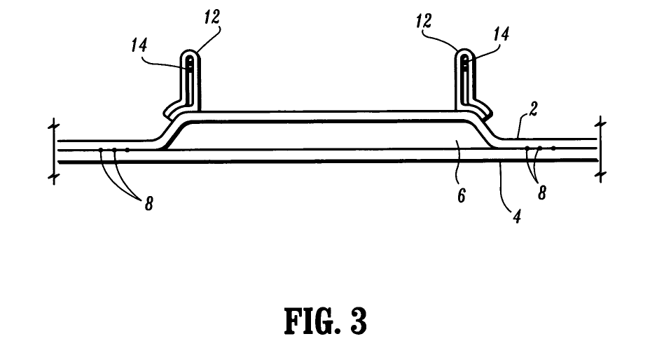

As best shown in FIGS. 2 and 3, the waste containment flaps 12 may be formed from separate elasticized strips of material that are associated with the topsheet 2, backsheet 4 or both, or otherwise integrated into the garment. In another preferred embodiment, the topsheet 2 and backsheet 4 have similar dimensions or different dimensions, but in either case, the waste containment flaps 12 are attached to the topsheet 2 or to some intermediate element which in turn is attached to the topsheet 2. The waste containment flaps 12 may be treated with a suitable surfactant to modify their hydrophobicity or hydrophilicity or imbued with skin wellness products as desired. Each waste containment flap 12 preferably includes a portion that folds over onto itself to form an enclosure. One or more elastic members 14 may be secured in the enclosure in a stretched condition. Various other configurations of topsheets 2 and waste containment systems, such as flaps 12, are known in the art, and the present invention is not intended to be limited to any particular design for these components.

Each leg opening 28a, 28b may be provided with a leg elastic containment system 9, sometimes referred to as conventional leg gathers. In an illustrative embodiment, three strands of elastic threads 8 are positioned to extend adjacent each leg openings 28a, 28b between the topsheet 2 and the backsheet 4. The selection of appropriate elastics and the construction of leg elastic containment systems are known in the art. For example, the leg elastics 8 may be ultrasonically bonded, heat and pressure sealed using a variety of bonding patterns, or glued to the diaper 10. Leg gathers 9 may be formed with or as separate members from topsheet 2.

Various commercially available materials may be used for the leg elastics 8 and elastic members 14, such as natural rubber, butyl rubber or other synthetic rubber, urethane, elastomeric materials such as spandex, which is marketed under various names, including LYCRA® (DuPont), GLOSPAN™ (Globe) and SYSTEM 7000™ (Fulflex). The present invention is not limited to any particular elastic material or to any particular shape, size or number of elastics.

The underlying structure beneath the topsheet 2 may include, depending on the absorbent garment construction, various combinations of elements, but in each embodiment, it is contemplated that the absorbent garment preferably will include an absorbent core 6. Although the absorbent core 6 depicted in FIG. 2 has a substantially rectangular shape as viewed in the plan view, other shapes may be used, such as a “T” shape or an hourglass shape. The absorbent core 6 may extend into either or both of the front and back waist regions 22, 24. The shape and construction of the absorbent core 6 may be selected to provide the greatest absorbency in target areas where body fluids are most likely to strike the diaper 10, which is often referred to as zoned absorbency. The absorbent core 6 may also comprise a number of layers of similar or different construction. The absorbent core may be associated with the topsheet 2, backsheet 4 or any other suitable part of the garment 10 by any method known in the art, in order to fix the absorbent core 6 in place.

Generally, in an illustrative embodiment, the absorbent core 6 comprises particles of super absorbent material (SAP) distributed within a fibrous structure. Additional fibrous or particulate additives may be disposed within the absorbent core 6 to add to the core's strength and SAP efficiency or to otherwise enhance the performance of the garment. The absorbent core 6 may be partially or wholly surrounded by additional layers (not shown) added to provide further benefits. The additional layer or layers may comprise any useful layer known in the art or developed hereafter, such as a fluid acquisition layer, a distribution layer, an additional fibrous layer optionally containing super absorbent particles, a wicking layer a storage layer, or combinations and fragments of these layers. Such layers may be provided to assist with transferring fluids to the absorbent core 6, handling fluid surges, preventing rewet, containing absorbent material, improving core stability or for other purposes.

The absorbent core 6 may be made from any absorbent material or materials, or combinations of such materials, known in the art or hereafter discovered. In one embodiment of the invention, the absorbent core 6 comprises wood fibers or other fibers such as chemical wood pulp, fibrous absorbent gelling material or any other suitable liquid absorbing material, such as commercially available fluff pulp or fluffed bleached kraft softwood pulp or fibrous absorbent gelling material. In another embodiment of the invention, the absorbent core 6 comprises a combination of a porous fibrous web and super absorbent particles. Absorbent cores are known in the art and exemplary cores are disclosed, for example, in U.S. Pat. No. 5,281,207 issued to Chmielewski et al., U.S. Pat. No. 4,610,678 issued to Weisman et. al., U.S. Pat. No. 5,137,537 issued to Herron et. al., U.S. Pat. No. 5,147,345 issued to Young et. al., and U.S. Pat. No. 6,068,620 issued to Chmielewski, all of which are incorporated herein by reference in their entirety, and in a manner consistent with the present invention.

Preferably, the absorbent core is thin in order to improve the comfort and appearance of a garment. The importance of thin, comfortable garments is disclosed, for example, in U.S. Pat. No. 5,098,423 to Pieniak et al., which is incorporated herein by reference in its entirety and in a manner consistent with the present invention.

Referring to FIG. 2, diaper 10 is fastened onto a wearer by using one or more, and preferably two, fastener tabs or fasteners 32. Fastener tabs 32 are preferably affixed to the chassis of the diaper 10 to extend laterally outward (i.e., in the lateral direction 102) from a waist region 22, 24 of the garment. The fasteners 32 preferably are positioned to extend outward from the ear portions 46 of the rear waist region 24, but the fastener 32 may also be attached to extend outward from the front waist region 22, or from both waist regions. The fastener 32 may extend from one, but preferably both, lateral sides of the diaper 10. The fasteners 32 may be attached to any part of the diaper chassis, such as topsheet 2, backsheet 4, outer cover or other layer of the diaper. The fastener 32 may also be attached to either side of the diaper's chassis, to multiple layers of the chassis or may be sandwiched between the various sheets comprising the chassis of the diaper 10. For example, fastener 32 may be positioned between topsheet 2 and backsheet 4 or to the topsheet 2 and backsheet 4 independently. Variations on the number, location and attachment configuration of the fasteners 32 will be apparent to those skilled in the art based on the teachings herein, and all such variations are within the scope of the present invention. It is contemplated herein that fasteners 32 may be formed with materials that are elastic, non-elastic or a combination thereof, include one or more substrate layers and may be attached to the diaper 10 or each other by adhesive, ultrasonic, thermal bonding or the like.

With reference to FIGS. 2, 4, 7 and 8, a grip 72 is attached, either directly or indirectly, at or near a distal portion of fastener 32. Grip 72 may comprise any hook-and-loop type fastener, adhesive fastener, or other type of fastener that is capable of holding the diaper 10 on a wearer. Suitable hook-and-loop fasteners are readily available from 3M Corp. (St. Paul, Minn.), Aplix, Inc. (Charlotte, N.C.), and Velcro USA, Inc. (Manchester, N.H.). Adhesive fasteners are also readily available, and disclosed, for example, in U.S. Pat. No. 4,645,501 issued to Teed, which is incorporated herein by reference in its entirety and in a manner consistent with the present invention. Suitable grips 72 of these and other types are generally known in the art and the present invention is not limited to the use of any particular type of grip 72.

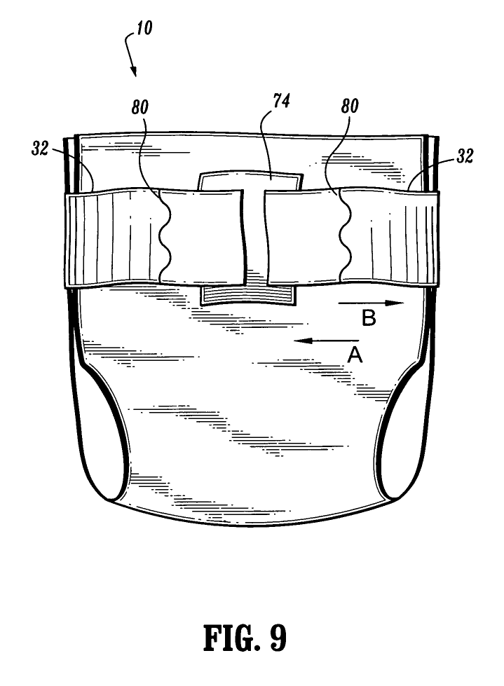

The grip 72 operates by engaging with or adhering to a corresponding surface, object or landing zone 74 (FIG. 9) located on the opposite end of the diaper 10, preferably front waist region 22. In those cases in which a hook-and-loop type fastening system is used, the hook portion of the system preferably is used as the grip 72, and the loop portion preferably is affixed to or contained within the outer surface of the diaper 10 at the end opposite the fastener tabs 32. An integral portion of the diaper 10, such as the backsheet 4 or an outer covering, may also be adapted to serve as the loop portion or other object to which the grip 72 attaches, either in its normal state or after being treated to better engage with the grip 72. The design and selection of a surface or object to which the grip 72 fastens or attaches is known in the art, and the present invention is not limited to the use of any particular device or construction for these parts.

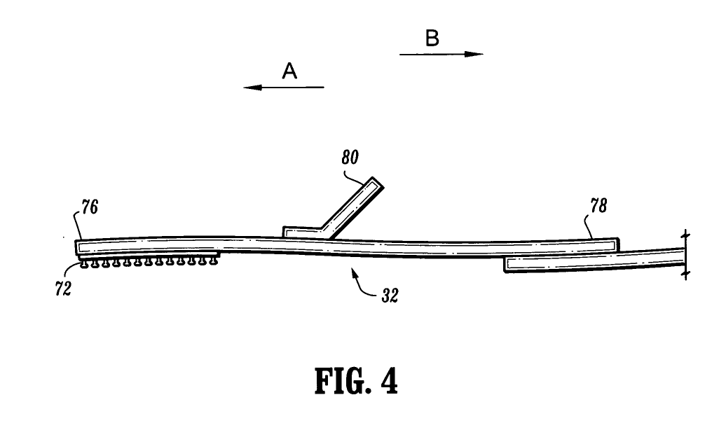

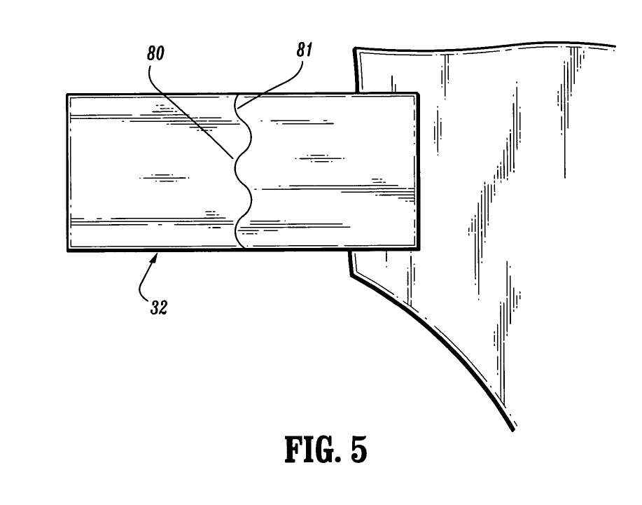

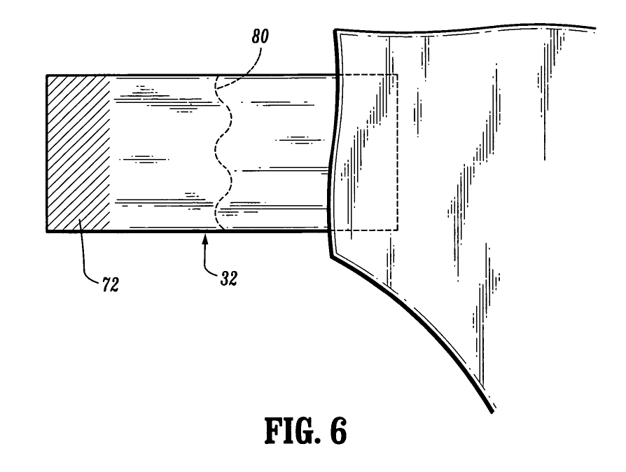

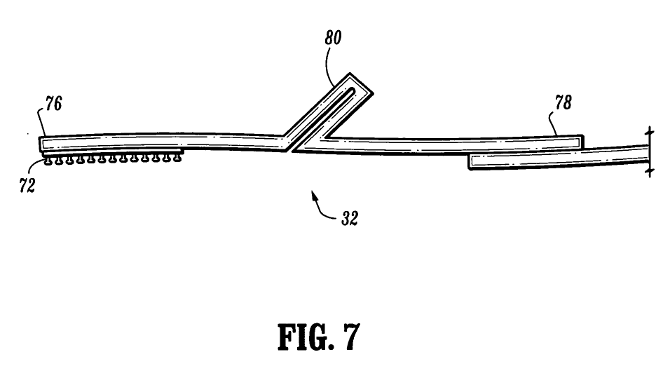

With reference to FIGS. 4 and 7, fastener 32 includes a first or distal end 76 and a second or proximal end 78. Fastener 32 includes a means for unfastening or disengaging, such as lifting region or finger lift 80 that is disposed intermediate the distal and proximal ends 76, 78. Finger lift 80 is adapted to allow the fastener 32 to disengage from front waist region 22 by, for example, lifting or pulling finger lift 80 towards distal end 76 (i.e., in a direction A as shown in FIG. 9). In addition, finger lift 80 is positioned such that fastener 32 does not disengage when finger lift 80 travels towards proximal end 78 (i.e., in a direction B as shown in FIG. 9). Finger lift 80 can have a wavy edge 81, or alternatively, another type of curved edge, or a straight edge (FIGS. 5 and 6). With reference to FIG. 4, a finger lift 80 can be formed as a separate member that is attached (by way of adhesive, ultrasonic, thermal bonding or the like) to a fastener 32 along a mid-portion or mid-length that is situated central to the distal and proximal ends of the fastener. Moreover, as is shown in FIG. 7, a finger lift 80 can also be formed by folding a portion of fastener 32. The folded portion of finger lift 80 may be formed by connecting one or more portions of fastener 32 to each other (by way of adhesive, ultrasonic, thermal bonding or the like) along a mid-portion or mid-length that is situated central to the distal and proximal ends of the fastener.

In contrast, a prior art fastener 100 shown in FIGS. 1A, 1B and 1C includes a lift region 102 disposed at an edge of the fastener 100. Consequently, a child playfully pulling lift region 102 in a proximal direction B may unfasten fastener 100 and thus remove the diaper 104 at an inappropriate time.

With particular reference to FIGS. 4-9, the child-resistant closure system of the present disclosure includes a tab or finger lift member 80. Finger lift 80 may be situated along any portion or sides of the fastener 32. In one embodiment, the finger lift 80 is generally situated along a central portion 94 of the fastener 32. In this configuration, movement of the finger lift 80 in the A direction (FIG. 9) provides for increased resistance and difficulty of separation of the fastener 32 from the landing zone 74. Similarly, movement of the finger lift 80 in the B direction provides for less resistance and facilitates detachment of the fastener 32 from the landing zone 74. Consequently, a wearer (i.e., a child, for example) engaging the finger lift 80 and providing a force in any direction not in the B direction would be met with resistance that may not succeed in detaching the fastener 32 from the landing zone 74.

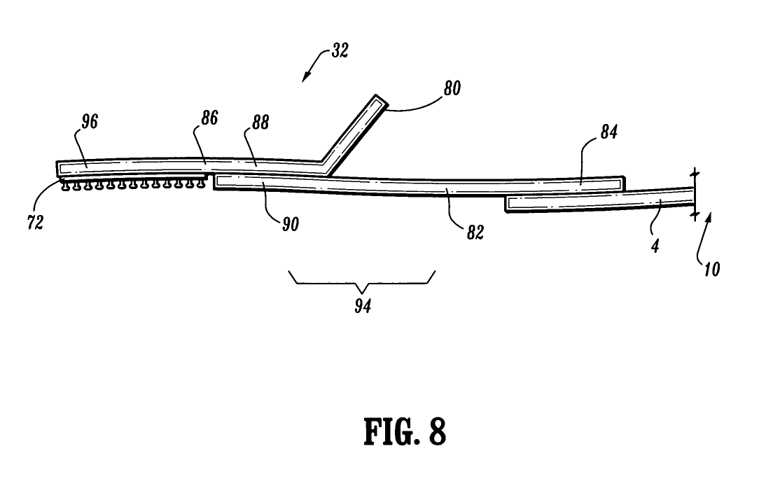

With attention to FIG. 8, an embodiment of the present disclosure provides fastener 32 having a first substrate 82 attached along its proximal end 84 to the diaper 10. Fastener 32 further includes a second substrate 86 disposed with the first substrate 82. More specifically, second substrate 86 is attached to the first substrate 82 generally along a mid-portion 88 that is generally central of the distal and proximal ends of the fastener 32. Mid-portion 88 is generally attached to a distal end 90 of first substrate 82. Second substrate 86 includes a distal portion 96 that is engageable with the front waist region 22 of the diaper 10. Second substrate 86 further includes a proximal portion disposed along a mid-portion 94 of the fastener 32. The mid-portion region 94 of the fastener 32 includes a detachment tab 80. Although, the detachment tab 80 is shown attached to second substrate 86 and situated along a mid-portion of the fastener 32, it is contemplated herein that the detachment tab 80 may be formed separately and otherwise attached to the fastener 32 or part of the first or second substrate 82, 86.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A fastener comprising:
a first end and a second end; and
means for disengagement disposed intermediate the first and second ends.

2. The fastener as recited in claim 1, wherein the fastener is disengaged by actuation of the means for disengagement generally in the direction of the first end.

3. The fastener as recited in claim 1, wherein actuation of the means for disengaging generally in the direction of the second end does not disengage the fastener.

4. The fastener as recited in claim 1, wherein the fastener is a substrate having a first side and a second side, and the means for disengagement protrudes from the first side.

5. The fastener as recited in claim 1, wherein the means for disengagement is attached to a mid-portion of the fastener.

6. The fastener as recited in claim 1, wherein the fastener includes a width, and wherein the means for disengagement includes a width substantially equal to that of the width of the fastener.

7. The fastener as recited in claim 1, wherein the means for disengagement is formed by a folded portion of the fastener.

8. The fastener as recited in claim 1, wherein the means for disengagement includes a finger lift portion that is attached to the fastener.

9. The fastener as recited in claim 1, wherein the fastener includes a plurality of substrates, and wherein the means for disengagement is formed by at least one of the plurality of substrates.

10. A diaper comprising:
a backsheet, a topsheet, and an absorbent core disposed therebetween, the diaper extending from a front end to a back end;
a fastener attached to the back end and being engageable with the front end for disposing the diaper about a subject, the fastener including at its mid-length a finger lift for disengaging the fastener from the front end when the finger lift is actuated in an inward direction.

11. The diaper as recited in claim 10, wherein actuation of the finger lift includes lifting or pulling the finger lift in an inward direction for disengaging the fastener from the front end.

12. The diaper as recited in claim 10, wherein actuation of the finger lift in an outward direction does not disengage the fastener from the front end.

13. The diaper as recited in claim 10, wherein the fastener is a substrate having a first side and a second side, and the finger lift is attached to the first side.

14. The diaper as recited in claim 10, wherein the finger lift is formed by a folded portion of the fastener.

15. The diaper as recited in claim 10, wherein a distal edge of the fastener includes a wavy configuration.

16. A diaper comprising:
a backsheet, a topsheet, and an absorbent core disposed therebetween, the diaper extending from a front end to a back end; and
a fastener distally attached to the back end and being engageable with the front end for disposing the diaper about a subject, the fastener including:
a first substrate having a proximal end attached to the back end of the diaper;
a second substrate attached to the first substrate;
a distal portion for engaging the front end of the diaper; and
a centrally disposed portion forming a means for disengagement of the fastener from the front end of the diaper.

17. The diaper as recited in claim 16, wherein the distal portion includes hook material.

18. The diaper as recited in claim 16, wherein a distal edge of the second substrate is co-extensive with the hook material.

19. The diaper as recited in claim 18, wherein the distal edge extends beyond the hook material.

20. The diaper as recited in claim 16, wherein an edge portion of the means of disengagement is non-straight.

21. The diaper as recited in claim 16, wherein an edge portion of the means of disengagement is wavy.
